(19) United States
(12) Patent Application Publication
(10) Pub. No.: US 2025/0058036 A1
(43) Pub. Date: Feb. 20, 2025

(54) SELF-CALIBRATION WITH DYNAMIC THERAPY PERFORMANCE FOR NEGATIVE PRESSURE WOUND THERAPY DEVICES

(71) Applicant: T.J.Smith and Nephew,Limited, Hull (GB)

(72) Inventor: Felix Clarence Quintanar, Hull (GB)

(21) Appl. No.: 18/720,399

(22) PCT Filed: Jan. 11, 2023
(86) PCT No.: PCT/EP2023/050559

§ 371 Date: Jun. 14, 2024

(30) Foreign Application Priority Data
Jan. 14, 2022 (GB) 2200446.9

(51) Int. Cl. A61M 1/00; A61M 39/24

(52) U.S. Cl. CPC A61M 1/962; A61M 1/966; A61M 1/985; A61M 39/24; A61M 2205/10; A61M 2205/3327; A61M 2205/3334; A61M 2205/50; A61M 2205/8212

(57) ABSTRACT

Disclosed is a negative pressure wound therapy system with self-calibration and safety features responsive to changes in the altitude. A wound therapy device can be configured to apply negative pressure to a wound via a negative pressure source. A pair of pressure sensors or a differential pressure sensor can monitor pressure in a fluid flow path and the negative pressure source can be controlled based on the monitored pressure. Self-calibration can include varying an activity level of the negative pressure source to reach a negative pressure set point and obtain a reference activity level, which can be used during subsequent activations of the negative pressure source. Changes in the altitude can be monitored and one or more operational parameters can be adjusted.

### TECHNICAL FIELD

Embodiments described herein relate to apparatuses, systems, and methods the treatment of wounds, for example using dressings in combination with negative pressure wound therapy.

### DESCRIPTION OF THE RELATED ART

The treatment of open or chronic wounds that are too large to spontaneously close or otherwise fail to heal by means of applying negative pressure to the site of the wound is well known in the art. Negative pressure wound therapy (“NPWT”) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

### SUMMARY

A negative pressure wound therapy system can include a wound dressing configured to be placed over a wound and absorb fluid aspirated from the wound. The system can include a negative pressure source configured to aspirate fluid from the wound via a fluid flow path connecting the negative pressure source to the wound dressing. The system can include a non-return valve positioned in the fluid flow path and fluidically connected to the negative pressure source. The system can include an electronic control circuitry configured to, based on a pressure difference between a first pressure in the fluid flow path and a second pressure of an environment surrounding the wound, activate the negative pressure source to establish a target negative pressure level in the fluid flow path. The electronic control circuitry can be configured to determine an activity level of the negative pressure source required to open and maintain the non-return valve in an opened state and establish the target negative pressure level in the fluid flow path. The electronic control circuitry can be configured to use the activity level during one or more subsequent activations of the negative pressure source to establish the target negative pressure level in the fluid flow path.

The negative pressure wound therapy system of any of the preceding paragraphs and/or any of the apparatuses, systems, or devices disclosed herein can include one or more of the following features. The electronic control circuitry can be configured to determine the activity level of the negative pressure source responsive to initially establishing the target negative pressure level in the fluid flow path. The electronic control circuitry can be configured to use the activity level for maintaining the target negative pressure level in the fluid flow path following initial establishment of the target negative pressure level in the fluid flow path. The electronic control circuitry can be configured to deactivate the negative pressure source responsive to initially establishing the target negative pressure level in the fluid flow path and subsequently activate the negative pressure source responsive to negative pressure in the fluid flow path becoming more positive than the target negative pressure level.

The negative pressure wound therapy system of any of the preceding paragraphs and/or any of the apparatuses, systems, or devices disclosed herein can include one or more of the following features. The electronic control circuitry can be configured to monitor a change in the second pressure of the environment surrounding the wound. The electronic control circuitry can be configured to responsive to determining that the second pressure is increasing, deactivate the negative pressure source to prevent reducing pressure at the wound to a negative pressure level associated with unsafe negative pressure. The electronic control circuitry can be configured to responsive to determining that the second pressure is decreasing, reduce the activity level of the negative pressure source until the pressure of the environment surrounding the wound has stabilized. The electronic control circuitry can be configured to responsive to determining that the change in the second pressure satisfies a threshold indicative or an abrupt change in pressure, deactivate the negative pressure source until the second pressure has stabilized. The electronic control circuitry can be configured to record a reference value of the second pressure responsive to activating the negative pressure source to establish the target negative pressure level in the fluid flow path and use the reference value of the second pressure to monitor the change in the second pressure.

The negative pressure wound therapy system of any of the preceding paragraphs and/or any of the apparatuses, systems, or devices disclosed herein can include one or more of the following features. The system can include a first absolute pressure sensor configured to monitor the first pressure in the fluid flow path. The system can include a second absolute pressure sensor configured to monitor the second pressure of the environment surrounding the wound. The electronic control circuitry can be configured to determine the pressure difference based on the first pressure monitored by the first absolute pressure sensor and the second pressure monitored by the second absolute pressure sensor.

The negative pressure wound therapy system of any of the preceding paragraphs and/or any of the apparatuses, systems, or devices disclosed herein can include one or more of the following features. At least one of the negative pressure source or the electronic circuitry can be disposed on or within the wound dressing. The system can include a differential pressure sensor configured to monitor the pressure difference between the first pressure in the fluid flow path and the second pressure of the environment surrounding the wound. The differential pressure sensor may not reference atmospheric pressure. The electronic control circuitry can be configured to determine the activity level responsive to calibration of the system and use the activity level for aspirating fluid from the wound of a patient. Calibration can be performed during manufacturing. The activity level can be a duty cycle of the negative pressure source. The system can include a power source configured to provide power to the negative pressure source and the electronic control circuitry. The electronic control circuitry can be configured to operate the negative pressure source by alternately activating and deactivating the negative pressure source to preserve capacity of the power source.

Disclosed herein are methods of operating a negative pressure wound therapy device of any of the preceding paragraphs and/or any of the devices, apparatuses, or systems disclosed herein.

A method of operating a negative pressure wound therapy system can include based on a pressure difference between a first pressure in a fluid flow path connecting a negative pressure source of the system to a wound covered by a wound dressing and a second pressure of an environment surrounding the wound, activating the negative pressure source to establish a target negative pressure level in the fluid flow path. The method can include determining an activity level of the negative pressure source required to open and maintain in an open state a non-return valve of the system and establish the target negative pressure level in the fluid flow path. The method can include using the activity level, establishing the target negative pressure level in the fluid flow path during one or more subsequent activations of the negative pressure source. The method can be performed by an electronic control circuitry of the system.

The method of any of the preceding paragraphs and/or any of the methods disclosed herein can include one or more of the following features. The method can include determining the activity level of the negative pressure source responsive to initially establishing the target negative pressure level in the fluid flow path. The method can include using the activity level for maintaining the target negative pressure level in the fluid flow path following initial establishment of the target negative pressure level in the fluid flow path. The method can include monitoring a change in the second pressure of the environment surrounding the wound. The method can include responsive to determining that the second pressure is increasing, deactivating the negative pressure source to prevent reducing pressure at the wound to a negative pressure level associated with unsafe negative pressure. The method can include responsive to determining that the second pressure is decreasing, reducing the activity level of the negative pressure source until the pressure of the environment surrounding the wound has stabilized. The method can include responsive to determining that the change in the second pressure satisfies a threshold indicative or an abrupt change in pressure, deactivating the negative pressure source until the second pressure has stabilized.

The method of any of the preceding paragraphs and/or any of the methods disclosed herein can include one or more of the following features. The method can include recording a reference value of the second pressure responsive to activating the negative pressure source to establish the target negative pressure level in the fluid flow path and using the reference value of the second pressure to monitor the change in the second pressure. The method can include determining the activity level is performed responsive to calibration of the system during manufacturing. The method can include using the activity level for aspirating fluid from the wound of a patient.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIGS. 1A-1C illustrate a wound dressing incorporating a source of negative pressure and/or other electronic components within the wound dressing;

FIGS. 2A-2B illustrate an electronics unit that may be incorporated into a wound dressing;

FIG. 3 is an exploded perspective view of an electronics assembly enclosing an electronics unit within a housing;

FIG. 4A illustrates a bottom perspective view of the electronics assembly of FIG. 3;

FIG. 4B illustrates a top perspective view of the electronics assembly of FIG. 3;

FIG. 5A is an exploded view of a wound dressing incorporating an electronics assembly within the wound dressing layers;

FIG. 5B illustrates a cross sectional layout of the material layers of a wound dressing incorporating an electronics assembly within the dressing;

FIGS. 6A-6B and 7A-7B illustrate components of an electronics assembly;

FIG. 8 illustrates a pump exhaust mechanism;

FIGS. 9A-9B illustrate a bottom view and a top perspective view of an inlet protection mechanism, a negative pressures source, and an exhaust mechanism;

FIGS. 10A-10C illustrate a negative pressure wound therapy device.

FIGS. 11 and 12 illustrate processes for self-calibration of a wound therapy device.

FIG. 13 illustrates a plot of pressure over time during operation of a negative pressure wound therapy device.

FIGS. 14A and 14B illustrate plots of pressure differentials and activity during operation of a negative pressure wound therapy device.

FIG. 15 illustrates the process for responding to changes in external pressure.

### DETAILED DESCRIPTION

Embodiments disclosed herein relate to apparatuses and methods of treating a wound with reduced pressure, including a source of negative pressure and wound dressing components and apparatuses. These apparatuses and components, including but not limited to wound overlays, backing layers, cover layers, drapes, sealing layers, spacer layers, absorbent layers, transmission layers, wound contact layers, packing materials, fillers and/or fluidic connectors are sometimes collectively referred to herein as dressings.

It will be appreciated that throughout this specification reference is made to a wound. It is to be understood that the term wound is to be broadly construed and encompasses open and closed wounds in which skin may be torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in NPWT or topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, 1013.25 mbar, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (such as, −40 mmHg is less than −60 mmHg). Negative pressure that is “more” or “greater” than −X mmHg corresponds to pressure that is further from atmospheric pressure (such as, −80 mmHg is more than −60 mmHg). In some cases, local ambient atmospheric pressure is used as a reference point, and such local atmospheric pressure may not necessarily be, for example, 760 mmHg.

The negative pressure range can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the negative pressure apparatus.

### Wound Dressing

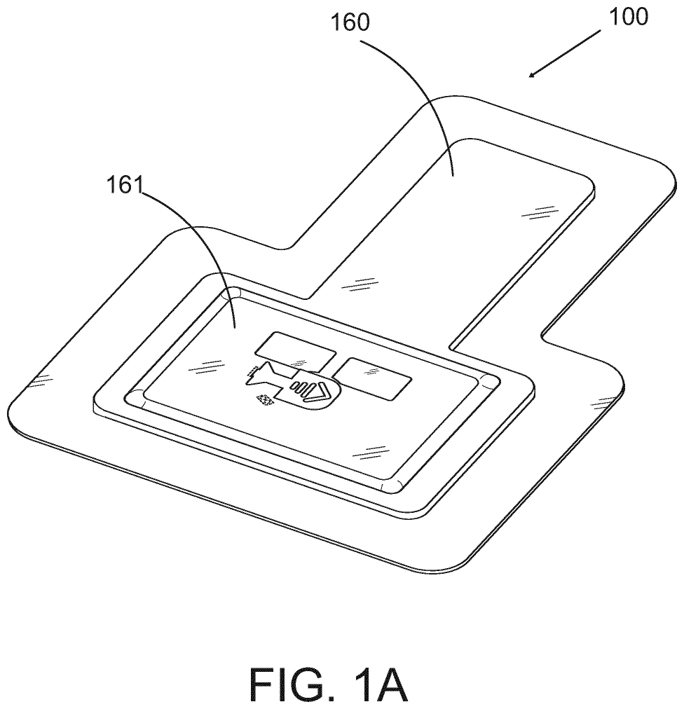

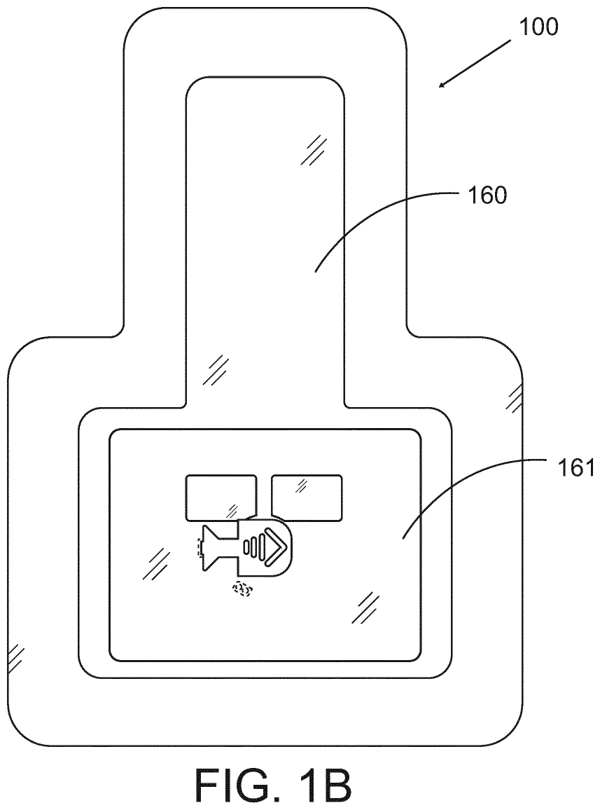

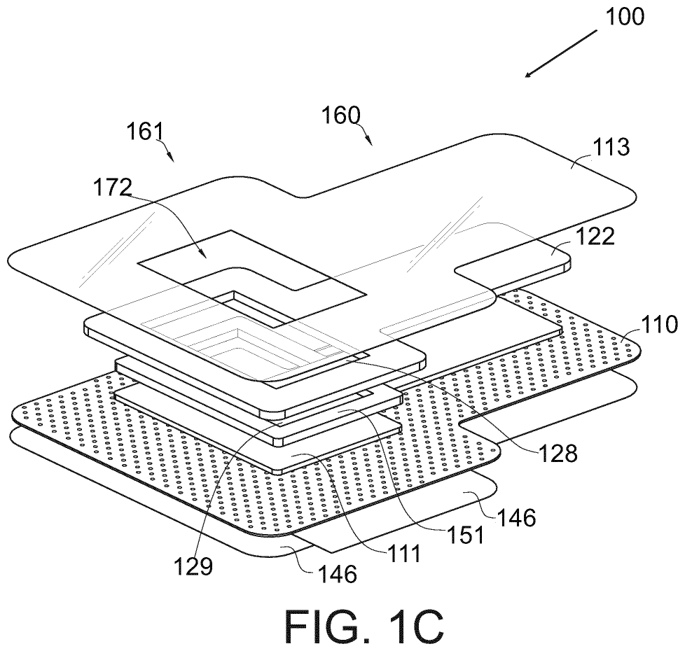

A source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The material layers can include a wound contact layer, one or more absorbent layers, one or more transmission or spacer layers, and a backing layer or cover layer covering the one or more absorbent and transmission or spacer layers. The wound dressing can be placed over a wound and sealed to the wound with the pump and/or other electronic components contained under the cover layer within the wound dressing. The dressing can be provided as a single article with all wound dressing elements (including the pump) pre-attached and integrated into a single unit. A periphery of the wound contact layer can be attached to the periphery of the cover layer enclosing all wound dressing elements as illustrated in FIG. 1A-1C.

The pump and/or other electronic components can be configured to be positioned adjacent to or next to the absorbent and/or transmission layers so that the pump and/or other electronic components are still part of a single article to be applied to a patient. The pump and/or other electronics can be positioned away from the wound site. Although certain features disclosed herein may be described as relating to systems and method for controlling operation of a negative pressure wound therapy system in which the pump and/or other electronic components are positioned in or on the wound dressing, the systems and methods disclosed herein are applicable to any negative pressure wound therapy system or any medical device. FIGS. 1A-1C illustrate a wound dressing incorporating the source of negative pressure and/or other electronic components within the wound dressing. FIGS. 1A-1C illustrate a wound dressing (or system) 100 with the pump and/or other electronics positioned away from the wound site. The wound dressing can include an electronics area 161 and an absorbent area 160. The dressing can comprise a wound contact layer 110 (not shown in FIGS. 1A-1B) and a moisture vapor permeable film, cover layer or backing layer 113 positioned above the contact layer and other layers of the dressing. The wound dressing layers and components of the electronics area as well as the absorbent area can be covered by one continuous cover layer 113 as shown in FIGS. 1A-1C.

A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. In particular, the transmission layer 111 preferably ensures that an open air channel can be maintained to communicate negative pressure over the wound area even when the absorbent layer has absorbed substantial amounts of exudates. The layer 111 should preferably remain open under the typical pressures that will be applied during negative pressure wound therapy as described above, so that the whole wound site sees an equalized negative pressure. The layer 111 may be formed of a material having a three dimensional structure. For example, a knitted or woven spacer fabric (for example Baltex 7970 weft knitted polyester) or a non-woven fabric could be used.

Further, one or more absorbent layers (such as layers 122, 151) for absorbing and retaining exudate aspirated from the wound can be utilized. A superabsorbent material can be used in the absorbent layers 122, 151. The one or more layers 122, 151 of absorbent material may be provided above the transmission layer 111. Since in use each of the absorbent layers experiences negative pressures, the material of the absorbent layer can be chosen to absorb liquid under such circumstances. The absorbent layers 122. 151 may comprise a composite comprising superabsorbent powder, fibrous material such as cellulose, and bonding fibers. The composite can be an airlaid, thermally-bonded composite.

The electronics area 161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 161 can include a button or switch (shown in FIGS. 1A-1B as being covered by a pull tab). The button or switch can be used for operating the pump (such as, turning the pump on/off).

The electronics area 161 of the dressing can comprise one or more layers of transmission or spacer material and/or absorbent material and electronic components can be embedded within the one or more layers of transmission or spacer material and/or absorbent material. The layers of transmission or absorbent material can have recesses or cut outs to embed the electronic components within whilst providing structure to prevent collapse. As shown in FIG. 1C, recesses 128 and 129 can be provided in absorbent layers 151 and 122, respectively.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

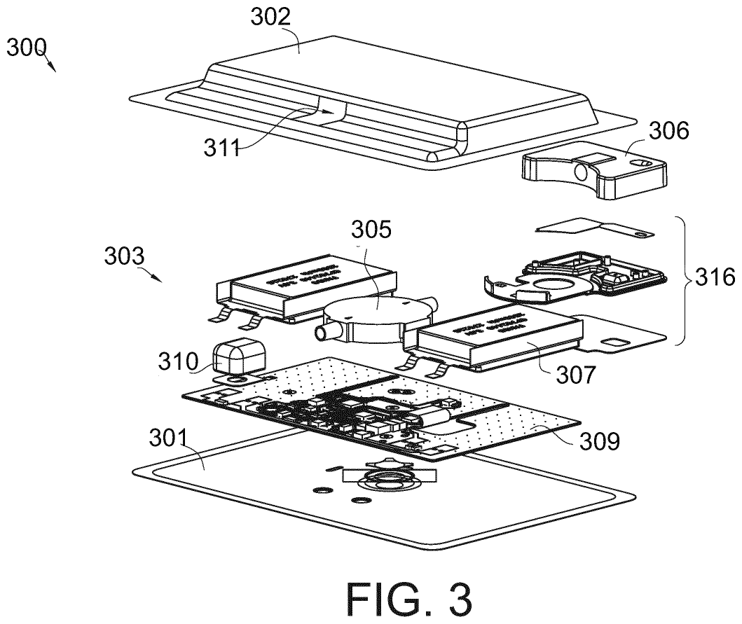

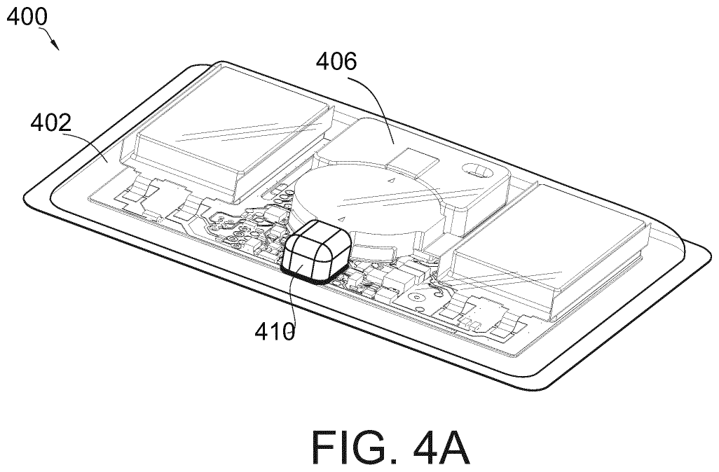

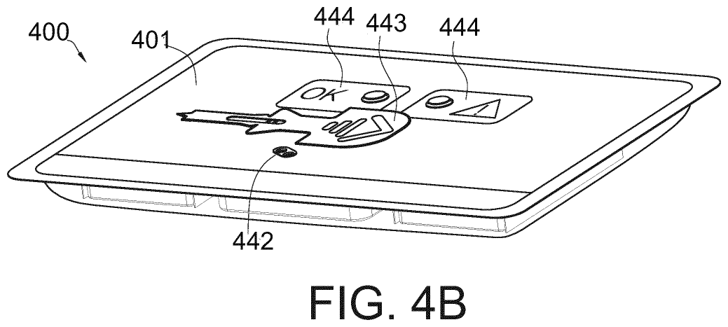

The cover layer may include a cutout 172 positioned over at least a portion of the aperture 128 in the absorbent layer 122 to allow access and fluid communication to at least a portion of the absorbent layers 122 and 151, transmission layer 111, and would contact layer 110 positioned below. An electronics assembly such as described below can be positioned in the apertures 128, 129, and 172 of the first and second absorbent material 151 and 122 and the cover layer 113. The electronics assembly can include a pump, power source, and a printed circuit board as described with reference to FIGS. 3 and 4A-4B.

Before use, the dressing can include one or more delivery layers 146 adhered to the bottom surface of the wound contact layer. The delivery layer 146 can cover adhesive or apertures on the bottom surface of the wound contact layer 110. The delivery layer 146 can provided support for the dressing and can assist in sterile and appropriate placement of the dressing over the wound and skin of the patient. The delivery layer 146 can include handles that can be used by the user to separate the delivery layer 146 from the wound contact layer 110 before applying the dressing to a wound and skin of a patient.

### Electronics Assembly Incorporated Within the Wound Dressing

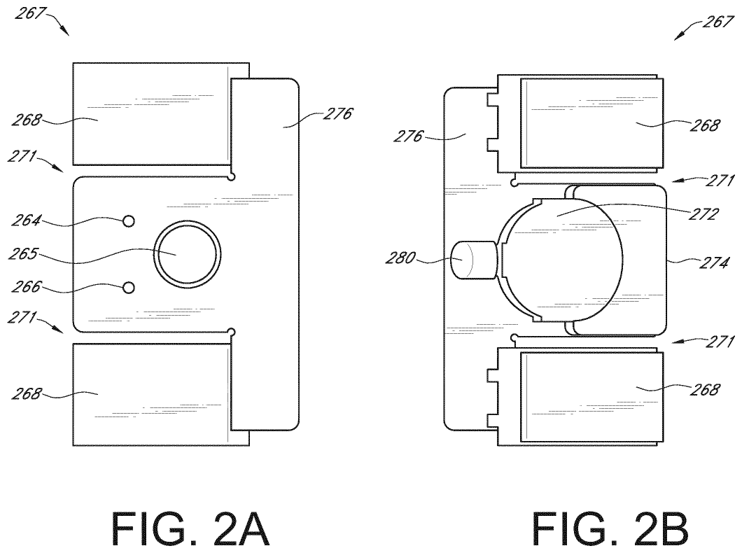

FIGS. 2A-2B illustrate an electronics unit 267 that can be incorporated into a wound dressing. FIG. 2A illustrates the top view of the electronics unit. FIG. 2B illustrates a bottom or wound facing surface of the electronics unit. The electronics unit 267 can include a pump 272 and one or more power sources 268, such as batteries. The electronics unit 267 can include a circuit board 276 configured to be in electrical communication with the pump 272 and/or power source 268. The circuit board 276 can be flexible or substantially flexible.

As illustrated in FIG. 2A, the electronics unit 267 can include single button or switch 265 on the upper surface of the unit. The single button or switch 265 can be used as an on/off button or switch to stop and start operation of the pump and/or electronic components. The electronics unit 267 can also include one or more vents or exhaust apertures 264 on the circuit board 276 for expelling the air exhausted from the pump. As shown in FIG. 2B, a pump outlet exhaust mechanism 274 (sometimes referred to as pump exhaust mechanism or pump outlet mechanism) can be attached to the outlet of the pump 272.

The electronics unit 267 can include a pump inlet protection mechanism 280 as shown in FIG. 2B positioned on the portion of the electronics unit closest to the absorbent area and aligned with the inlet of the pump 272. The pump inlet protection mechanism 280 is positioned between the pump inlet and the absorbent area or absorbent layer of the dressing. The pump inlet protection mechanism 280 can include hydrophobic material to prevent fluid from entering the pump 272. The pump inlet protection mechanism 280 (or any of the inlet protection mechanisms disclosed herein) can include a filter.

The upper surface of the electronics unit 267 can include one or more indicators 266 for indicating a condition of the pump and/or level of pressure within the dressing. The indicators can be small LED lights or other light source that are visible through the dressing components or through holes in the dressing components above the indicators. The indicators can be green, yellow, red, orange, or any other color. For example, there can be two lights, one green light and one orange light. The green light can indicate the device is working properly and the orange light can indicate that there is some issue with the pump (such as, leak, saturation level of the dressing, blockage downstream of the pump, exhaust blockage, low battery, or the like).

The power source 268 can be in electrical communication with the circuit board 276. One or more power source connections are connected to a surface of the circuit board 276. The circuit board 276 can have other electronics incorporated within. For example, the circuit board 276 may support various sensors including, but not limited to, one or more pressure sensors, temperature sensors, optic sensors and/or cameras, and/or saturation indicators.

FIG. 3 illustrates an electronics assembly 300 enclosing an electronics unit within a housing. As illustrated in FIG. 3, the housing of the electronics assembly 300 can include a plate 301 and flexible film 302 enclosing the electronics unit 303 within. The electronics unit 303 can include a pump 305, inlet protection mechanism 310, pump exhaust mechanism 306, power source 307, and circuit board 309. The circuit board 309 can be flexible or substantially flexible.

As is illustrated, the pump exhaust mechanism 306 can be an enclosure, such as a chamber. The electronics unit 303 and pump 305 can be used without the inlet protection mechanism 310. However, the pump exhaust mechanism 306 and the pump 305 can sit within an extended casing 316.

The flexible film 302 can be attached to the plate 301 to form a fluid tight seal and enclosure around the electronic components. The flexible film 302 can be attached to the plate at a perimeter of the plate by heat welding, adhesive bonding, ultrasonic welding, RF welding, or any other attachment or bonding technique.

The flexible film 302 can include an aperture 311. The aperture 311 can allow the inlet protection mechanism 310 to be in fluid communication with the absorbent and/or transmission layers of the wound dressing. The perimeter of the aperture 311 of the flexible film 303 can be sealed or attached to the inlet protection mechanism 310 to form a fluid tight seal and enclosure around the inlet protection mechanism 310 allowing the electronic components 303 to remain protected from fluid within the dressing. The flexible film 302 can be attached to the inlet protection mechanism 310 at a perimeter of the inlet protection mechanism 310 by heat welding, adhesive bonding, ultrasonic welding, RF welding, or any other attachment or bonding technique. The inlet protection mechanism 310 can prevent wound exudate or liquids from the wound and collected in the absorbent area 160 of the wound dressing from entering the pump and/or electronic components of the electronics assembly 300.

The electronics assembly 300 illustrated in FIG. 3 can be incorporated within the wound dressing such that, once the dressing is applied to the body of the patient, air from within the dressing can pass through the inlet protection mechanism 310 to be pumped out toward the pump exhaust mechanism 306 in communication with an aperture in the casing 316 and the circuit board 309 as described herein.

FIGS. 4A-B illustrate an electronics assembly 400 including a pump inlet protection mechanism 410 sealed to the exterior of the flexible film 402, similar to the description with reference to FIG. 3. Also shown is an exhaust mechanism 406, which can be similar to the exhaust mechanism 306.

FIG. 4A illustrates lower, wound facing surface of the electronics assembly 400. FIG. 4B shows an upper surface of the plate 401 (which can face the patient or user) of the electronics assembly 400. The upper surface of the plate 401 can include an on/off switch or button cover 443 (illustrated as a pull tab), indicators 444, and/or one or more vent holes 442. Removal of the pull tab 443 can cause activation of the electronics assembly 400, such as provision of power from the power source to the electronics assembly. Further details of operation of the pull tab 443 are described in PCT International Application No. PCT/EP2018/079745, filed Oct. 30, 2018, titled “SAFE OPERATION OF INTEGRATED NEGATIVE PRESSURE WOUND TREATMENT APPARATUSES,” which is incorporated by reference in its entirety herein.

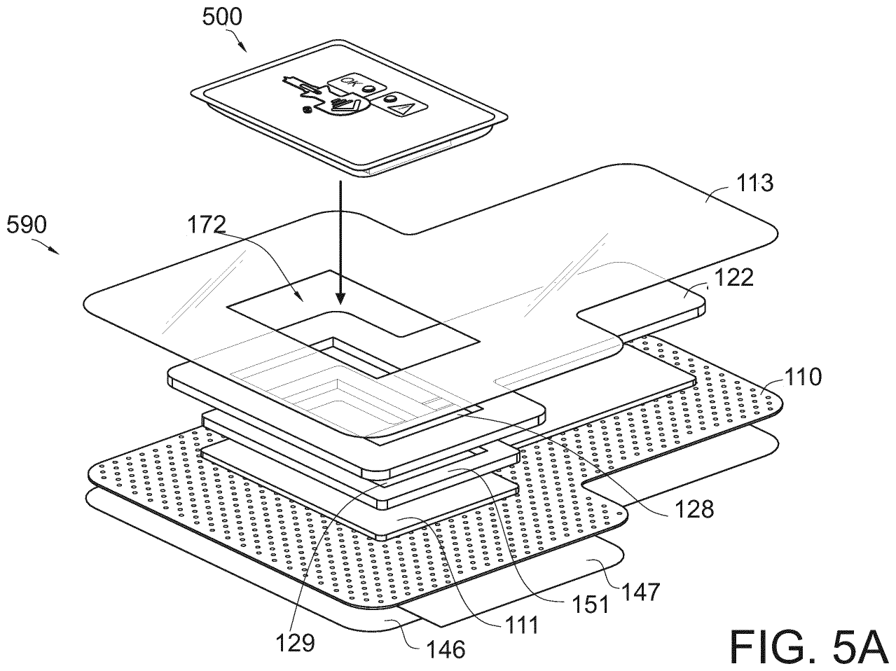

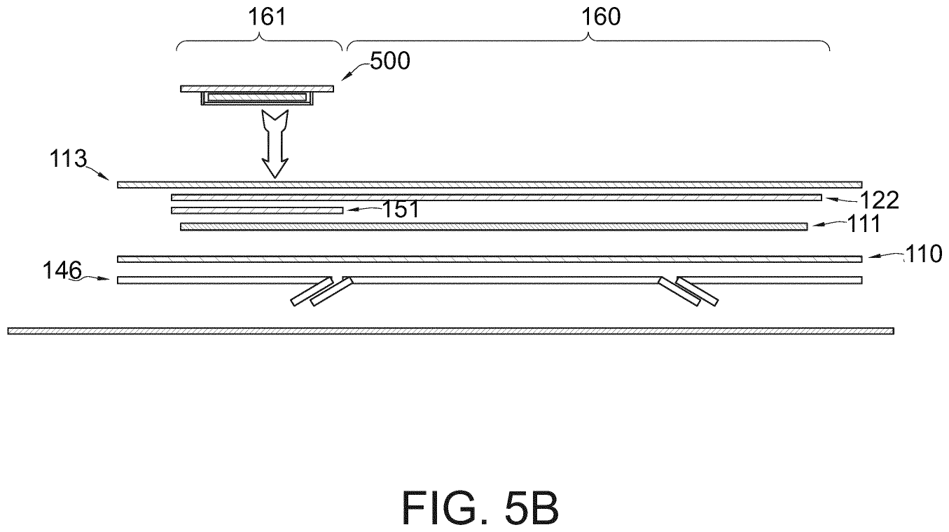

The electronics assembly 400 with the pump inlet protection mechanism 410 extending from and sealed to the film 402 can be positioned within the aperture 172 in the cover layer 113 and absorbent layer(s) (122, 151) as shown in FIG. 1C. The perimeter of the electronics assembly 400 can be sealed to a top surface of the outer perimeter of the aperture 172 in the cover layer 113 as shown in FIGS. 1C and described in more detail with reference to FIG. 5A-5B herein. The electronics assembly 400 can be sealed to the cover layer 113 with a sealant gasket, adhesive, heat welding, adhesive bonding, ultrasonic welding, RF welding, or any other attachment or bonding technique. The electronics assembly 400 can be permanently sealed to the cover layer 113 and could not be removed from the cover layer without destroying the dressing.

The electronics assembly 400 can be utilized in a single dressing and disposed of with the dressing. In some cases, the electronics assembly 400 can be utilized in a series of dressings.

FIG. 5A illustrates a wound dressing, such as the one in FIG. 1C, incorporating an electronics assembly 500 within the wound dressing layers 590. FIG. 5B illustrates a cross-sectional view of the wound dressing incorporating the electronics assembly of FIG. 5A. The electronics assembly 500 can be provided within the aperture 172 in the cover layer and apertures 129 and 128 in the first and second absorbent layers 122, 151. The electronics assembly 500 can seal to the outer perimeter of the aperture 172 of the cover layer. The dressing can comprise a wound contact layer 110 and a moisture vapor permeable film, cover layer or backing layer 113 positioned above the contact layer 110 and other layers of the dressing. A layer 111 of porous material can be located above the wound contact layer 110. As used herein, the terms porous material, spacer, and/or transmission layer can be used interchangeably to refer to the layer of material in the dressing configured to distribute negative pressure throughout the wound area. This porous layer, or transmission layer, 111 allows transmission of fluid including liquid and gas away from a wound site into upper layers of the wound dressing. Further, one or more absorbent layers (such as layers 122, 151) for absorbing and retaining exudate aspirated from the wound can be utilized. The one or more layers 122, 151 of absorbent material may be provided above the transmission layer 111. There may be a small aperatured absorbent layer 151 and a large aperture absorbent layer 122. The small apertured absorbent layer 151 can be positioned on top of the large apertured absorbent layer 122. In some cases, the small apertured absorbent layer 151 can be positioned below of the large apertured absorbent layer 122. Before use, the dressing can include one or more delivery layers 146 adhered to the bottom surface of the wound contact layer. The delivery layer 146 can cover adhesive or apertures on the bottom surface of the wound contact layer 110.

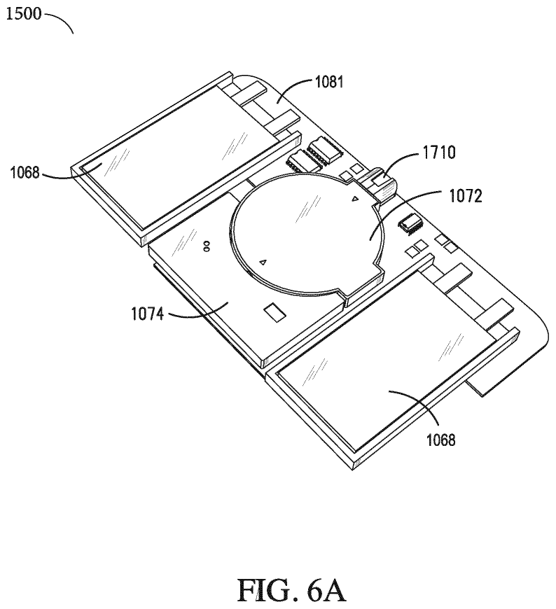

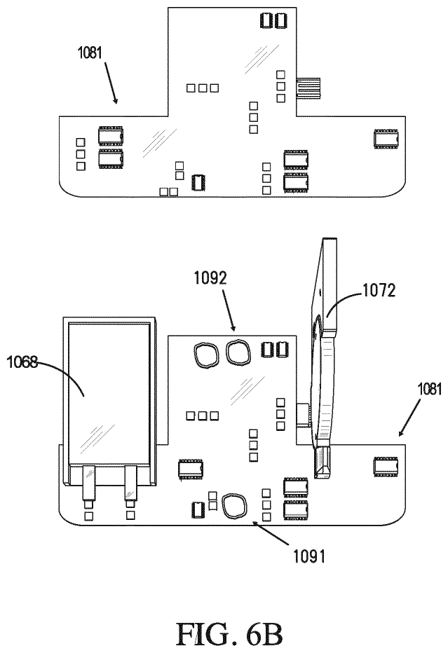

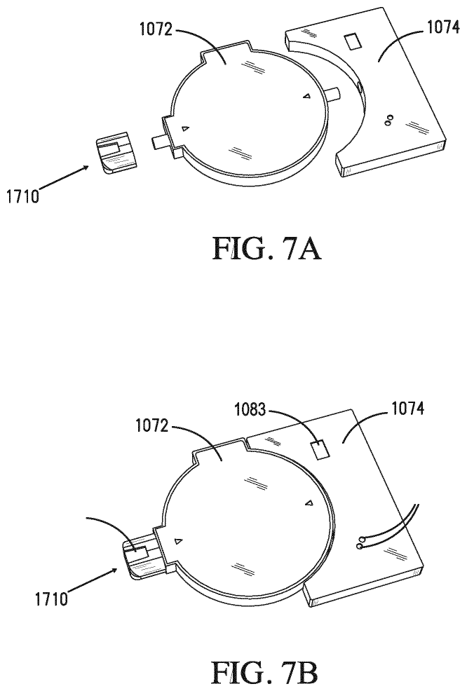

FIGS. 6A-6B and 7A-7B illustrate an electronics assembly 1500 with a pump inlet protection mechanism 1710 and pump exhaust mechanism 1074 on a pump 1072. The assembly 1500 can include cavities 1082 and 1083 (shown in FIGS. 7A-7B) on the pump inlet protection mechanism 1710 and pump exhaust mechanism 1074, respectively. The inlet protection and pump exhaust mechanisms can be adhered to the inlet and the outlet of the pump as described herein. The assembly 1500 can be assembled using an adhesive and allowed to cure prior to incorporating into the electronics assembly.

The pump inlet can be covered or fitted with a pump inlet protection mechanism 1710. The pump inlet protection 1710 can be pushed onto the pump inlet as illustrated by the arrows in FIG. 7A. This can be a friction fit. The port of the pump inlet protection 1710 that receives a portion of the pump inlet can be sized and shaped to be a complementary fit around the pump inlet. The pump inlet protection 1710 can be bonded onto the pump inlet using a silicone sealant or any other sealant or sealing technique. FIG. 7B illustrates the pump inlet protection mechanism 1710 covering the pump inlet and the pump exhaust mechanism 1074 covering the pump outlet. The pump exhaust mechanism 1074 can include one or more apertures or vents 1084 to allow gas aspirated by the pump to be exhausted from the pump exhaust mechanism 1074. In some cases, a non-return valve and/or filter membrane of the pump exhaust mechanism is included in the pump exhaust mechanism 1074.

FIGS. 7A-7B illustrate the pump inlet protection mechanism 1710 and pump exhaust mechanism 1074 with cavities 1082 and 1083. A pump assembly including the pump inlet protection mechanism 1710 and pump exhaust mechanism 1074 can be placed over the surface of a circuit board 1081. When the pump assembly is in contact with the surface of the circuit board 1081, the cavities 1082 and 1083 can at least partially enclose sensors on the circuit board 1081, for example, pressure sensors 1091 and 1092 on the circuit board 1081, as illustrated in FIG. 6B.

The pressure sensors 1091 and 1902 illustrated in FIG. 6B can be used to measure and/or monitor the pressure level at the wound and atmospheric pressure. The pressure sensor 1091 can be used to measure and/or monitor pressure at the wound (such as, underneath the wound dressing), which can be accomplished by measuring and/or monitoring pressure in a fluid flow path connecting the negative pressure source or pump 1072 and the wound. The pressure sensor 1091 can measure and/or monitor pressure in the cavity 1082 of the pump inlet protection mechanism 1710 shown in FIGS. 7A-7B. A power source 1068 (illustrated as two batteries in FIG. 6A) can provide power to the negative pressure source 1072 and the electronics.

The pressure sensor 1092 can be used to measure and/or monitor pressure external to the wound dressing. The pressure sensor 1092 can measure and/or monitor pressure in the cavity 1083 of the pump exhaust mechanism 1074 shown in FIGS. 7A-7B. The pressure sensor 1092 can measure pressure external to the wound dressing, which can be relative atmospheric pressure since the atmospheric pressure varies depending on, for instance, an altitude of use or pressurized environment in which the TNP apparatus may be used. These measurements can be used to establish a desired negative pressure differential (or set point) at the wound relative to the external pressure.

The circuit board 1081 (including any of the circuit boards described herein) can include control circuitry, such as one or more processors or controllers, that can control the supply of negative pressure by the negative pressure source 1072 according at least to a comparison between the pressure monitored by the pressure sensor 1091 and the pressure monitored by the pressure sensor 1092. Control circuity can operate the negative pressure source 1072 in a first mode (that can be referred to as an initial pump down mode) in which the negative pressure source 1072 is activated to establish the negative pressure set point at the wound. The set point can be set to, for example, a value in the range between about −70 mmHg to about −90 mmHg, among others. Once the set point has been established, which can be verified based on a difference between pressure measured by the pressure sensor 1091 (or wound pressure) and pressure measured by the pressure sensor 1092 (or external pressure), control circuitry can deactivate (or pause) operation of the negative pressure source 1072. Control circuitry can operate the negative pressure source 1072 is a second mode (that can be referred to as maintenance pump down mode) in which the negative pressure source 1072 is periodically activated to re-establish the negative pressure set point when the wound is depressurized as a result of one or more leaks. Control circuitry can activate the negative pressure source 1072 in response to the pressure at the wound (as monitored by the pressure sensor 1091) becomes more positive than a negative pressure threshold, which can be set to the same negative pressure as the set point or lower negative pressure.

Embodiments of the wound dressings, wound treatment apparatuses and methods described herein may also be used in combination or in addition to one or more features described in PCT International Application No. PCT/EP2017/060464, filed May 3, 2017, titled NEGATIVE PRESSURE WOUND THERAPY DEVICE ACTIVATION AND CONTROL, U.S. Pat. Nos. 8,734,425, and 8,905,985, each of which is hereby incorporated by reference in its entirety herein.

One or more self-adhesive gaskets can be applied to the pump inlet protection mechanism 1710 and pump exhaust mechanism 1074 to seal the cavities 1082 and 1083 of the pump inlet and pump exhaust around sensors on the circuit board 1081 and to seal around the exhaust mechanism vent(s) and corresponding vent(s) in the circuit board 1081 (as described herein). A pre-formed adhesive sheet can be used to form the sealing gaskets between the cavities 1082 and 1083 of the pump inlet and pump exhaust mechanisms and sensors on the circuit board 1081 and between the exhaust mechanism vent(s) and vent(s) in the circuit board 1081. In some cases, an adhesive can be used to seal the cavities 1082 and 1083 of the pump inlet protection 1710 and pump exhaust mechanism 1074 around sensors on the circuit board 1081 and to seal around the exhaust mechanism vent(s) 1084 and corresponding vent(s) in the circuit board. As described herein, the electronics assembly 1500 can be embedded within layers of the dressing, such as in cutouts or recesses into which the electronics assembly can be placed.

The pump inlet protection mechanism 1710 can provide a large surface area available for vacuum to be drawn by the inlet of the pump. A pump inlet (shown as rounded protrusion in FIG. 7A) can fit within a recess in the pump inlet protection mechanism 1710. The pump inlet can be attached by friction fit and/or form a complementary fit to the recess of the pump inlet protection mechanism.

The pump inlet protection mechanism 1710 can allow air or gas to pass through, but can block liquid from reaching the negative pressure source. The pump inlet protection mechanism 1710 can include a porous material. The pump inlet protection mechanism 1710 can comprise one or more porous polymer molded components. The pump inlet protection mechanism 1710 can include hydrophobic or substantially hydrophobic material. Material included in the pump inlet protection mechanism 1710 can have a pore size in the range of approximately 5 microns to approximately 40 microns. In some cases, the pore size can be approximately 10 microns. The pump inlet protection mechanism 1710 can include a polymer that can be one of hydrophobic polyethylene or hydrophobic polypropylene. In some cases, the pump inlet protection mechanism can include a Porvair Vyon material with a pore size of 10 microns. Any of the pump inlet protection mechanism described herein can include one or more features of the pump inlet protection mechanism 1710.

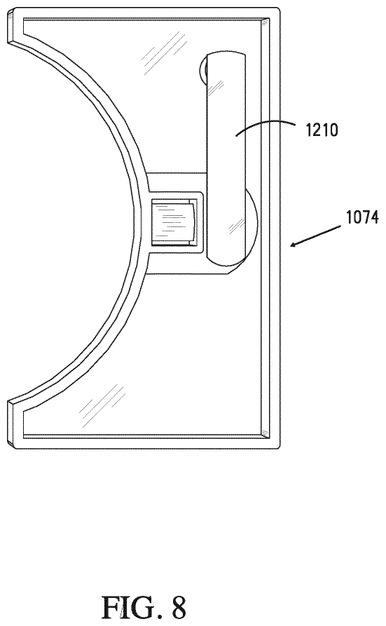

The pump exhaust mechanism 1074 (or any of the pump exhaust or outlet mechanisms described herein) can include a check valve, one-way, or a non-return valve 1210 as shown in FIG. 8. The non-return valve 1210 can be any suitable mechanical one-way valve, such as, for example, a reed valve, a duckbill valve, a ball valve, a loose leaf valve or an umbrella valve, among others. The non-return valve can be similar to any of the non-return valves described in PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING, which is incorporated by reference herein in its entirety. The pump exhaust mechanism 1074 can be bonded to the outlet of the pump using a sealant, for example a silicone sealant. The outlet or exhaust of the pump exhaust mechanism 1074 can include an antimicrobial film and/or other filter membrane that filters gas exhausted outside the NPWT system, such as to the atmosphere. As illustrated, pump exhaust mechanism 1074 can be an enclosure or chamber that is substantially sealed to prevent ingress of gas or fluid other than through the vent(s) 1084.

Any of the embodiments described herein can additionally or alternatively include one or more features described in International Application No. PCT/EP2018/074694, filed Sep. 13, 2018, titled NEGATIVE PRESSURE WOUND TREATMENT APPARATUSES AND METHODS WITH INTEGRATED ELECTRONICS, International Application No. PCT/EP2018/074701, filed Sep. 13, 2018, titled NEGATIVE PRESSURE WOUND TREATMENT APPARATUSES AND METHODS WITH INTEGRATED ELECTRONICS, International Application No. PCT/EP2018/079345, filed Oct. 25, 2018, titled NEGATIVE PRESSURE WOUND TREATMENT APPARATUSES AND METHODS WITH INTEGRATED ELECTRONICS, International Application No. PCT/EP2020/056317, filed Mar. 10, 2020, titled EXHAUST BLOCKAGE DETECTION FOR NEGATIVE PRESSURE WOUND TREATMENT APPARATUSES, each of which is incorporated by reference herein in its entirety.

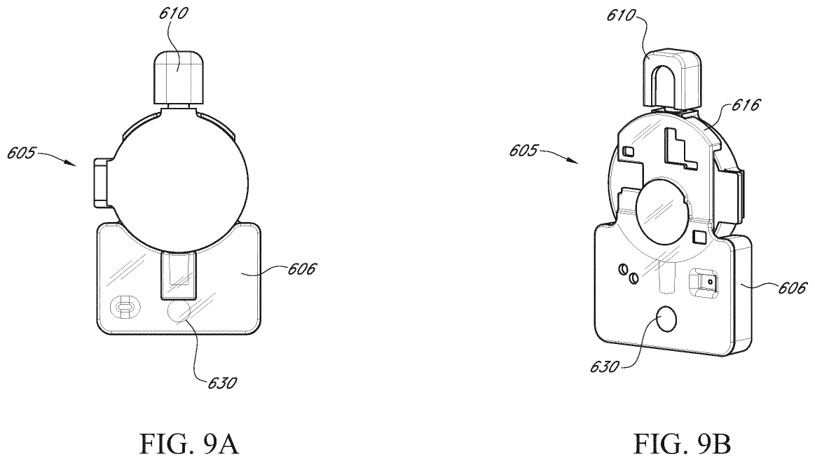

FIGS. 9A-9B illustrate bottom and top perspective views of a pump exhaust mechanism 606 (which can be any of the pump exhaust mechanisms described herein). The pump exhaust mechanism 606 can include at least one valve 630, which can be an umbrella valve. The at least one valve 630 can be configured to prevent or reduce the air from leaking through the pump exhaust mechanism 606 toward the dressing. As described herein, once the dressing is applied to the body of the patient, air from within the dressing can pass through an inlet protection mechanism 610 (which can be any of the inlet protection mechanisms described herein) to be pumped out toward the pump exhaust mechanism 606 in communication with an aperture in an extended casing 616. The at least one valve 630 can be configured to increase the efficiency of a pump 605 (which can be any of the pumps described herein).

### Canisterless Negative Pressure Wound Therapy Device

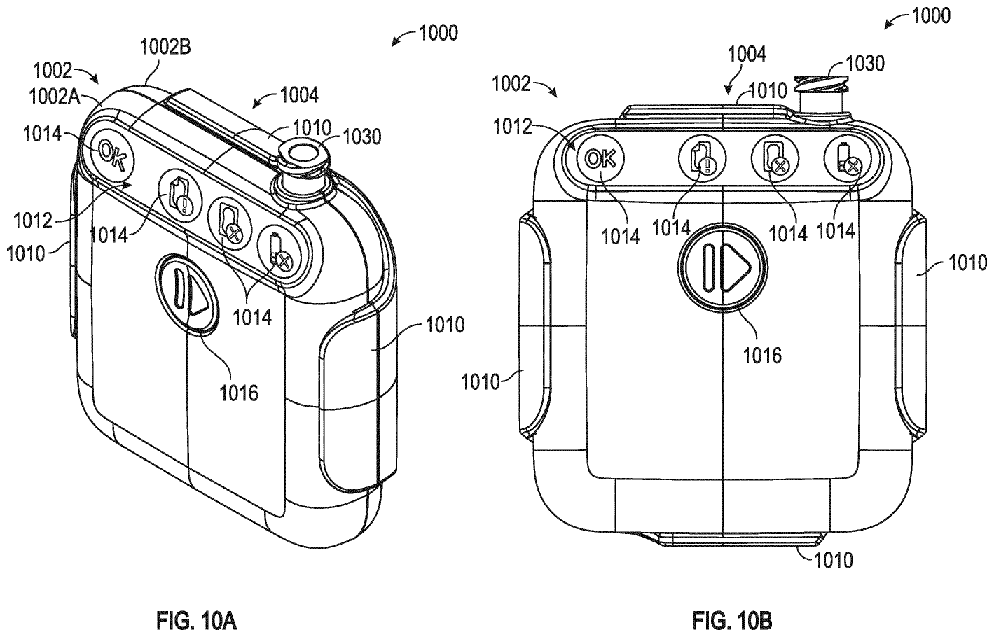

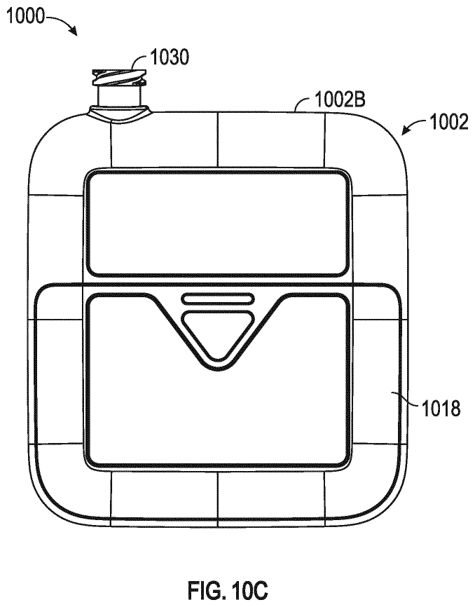

FIGS. 10A, 10B, and 10C illustrate perspective, front, and rear views of a negative pressure wound therapy device 1000 (sometimes referred to as a wound therapy device). The wound therapy device 1000 can include a housing 1002 and a mounting component 1010 (such as an attachment). The mounting component 1010 can be removably attached to the housing 1002, such that the wound therapy device 1000 can be used with or without the mounting component 1010. For example, FIG. 10C illustrates the wound therapy device 1000 without the mounting component 1010. The mounting component 1010 can be designed to allow the wound therapy device 1000 to be mounted on another object such as, but not limited to, a user's person. The mounting component 1010 can include a clip 1004 designed to retain the mounting component 1010 on a user's outerwear, such as on a user's pocket, a pouch, a belt, a flap, or otherwise.

The housing 1002 (sometimes referred to as “outer housing”) can contain or support components of the wound therapy device 1000. The housing 1002 can be formed from one or more portions, such as a front portion 1002A and a rear portion 1002B, which can be removably attached to form the housing 1002.

The housing 1002 can include a user interface 1012 which can be designed to provide a user with information (for example, information regarding an operational status of the wound therapy device 1000). The user interface 1012 can include one or more indicators, such as icons 1014, which can alert the user to one or more operating or failure conditions of the reduced pressure wound therapy system.

The wound therapy device 1000 can include one or more user input features, such as button 1016, designed to receive an input from the user for controlling the operation of the wound therapy device 1000. A single button can be present which can be used to activate and deactivate the reduced pressure wound therapy device or control other operating parameters of the wound therapy device 1000.

The wound therapy device 1000 can include a connector 1030 for connecting a tube or conduit to the wound therapy device 1000. The connector 1030 can be used to connect the wound therapy device 1000 to a wound dressing.

The wound therapy device 1000 can be a canisterless device. The wound dressing can retain fluid (such as, exudate) aspirated from the wound. Such a dressing can include a filter, such as a hydrophobic filter, that prevents passage of liquids downstream of the wound dressing (toward the wound therapy device 1000).

The wound therapy device 1000 can include a cover 1018, as illustrated in FIG. 10C and which can be removable. The cover 1018 can cover a cavity (not shown) in which one or more power sources, such as batteries, for powering the wound therapy device 1000 are positioned.

The wound therapy device 1000 can include one or more controllers or other electronic components described herein. The wound therapy device 1000 can be similar to the Pico negative pressure wound therapy device manufactured by Smith & Nephew.

Any of the negative pressure wound therapy devices described herein can include one or more features disclosed in U.S. Patent Publication No. 2019/0231939, which is incorporated by reference in its entirety.

### Self-Calibration Modes

Any of the negative pressure wound therapy systems or devices disclosed herein (such as, the system 100 or device 1000) can be configured to perform self-calibration. Self-calibration can be performed by any of the electronics units, controllers, processors, or assemblies disclosed herein (such as, the electronics unit 267, electronics assemblies 300, 400, or 500, circuit board 1081, or the electronics assembly 1500). A wound therapy system can be substantially sealed and operate without a source of reference gas flow from the external or surrounding environment (such as, reference atmospheric air). Such design may conserve the capacity of the power source since it may not be necessary to continuously operate a negative pressure source to maintain a desired negative pressure at the wound in the presence of the reference gas flow. However, due to changes in the external pressure (such as, atmospheric pressure), adjustment of operation may be advantageously performed based on data acquired during self-calibration. Self-calibration approaches described herein (such as, processes 1100 and 1200) can be particularly applicable to systems that utilize one or more absolute pressure sensors or one or more differential pressure sensors that do not directly reference external pressure. Such systems may not have direct access (or take direct measurement) of external pressure surrounding the system. As a result, changes in the external pressure due to, for instance, changes in elevation can undesirably affect the operation of the system unless such changes are accounted for.

Self-calibration can be performed to safely maintain a desired level of negative pressure at the wound regardless of the variance in the external pressure. Self-calibration can be implemented in hardware, software, or firmware. Advantageously, little additional hardware or no additional hardware may be needed and little or no modification to the existing electronics design of wound therapy systems may be needed. Static or dynamic optimization can be used to mitigate various factors (such as, component tolerance, parasitic characteristics, environmental changes, or the like) to improve the performance of the wound therapy device.

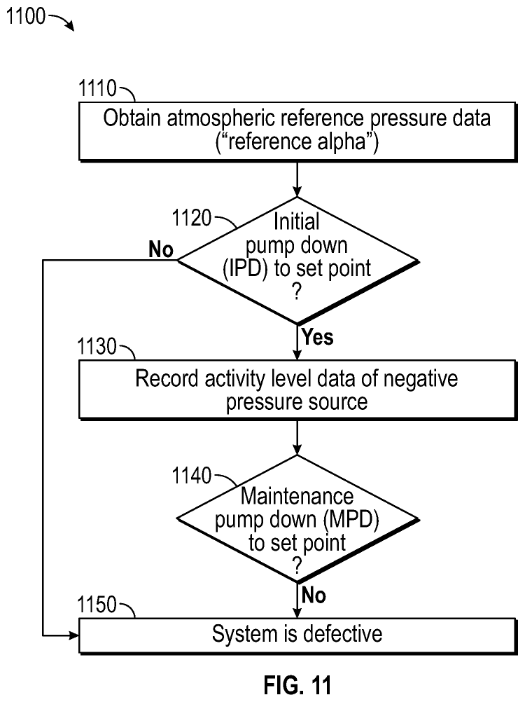

Referring to FIG. 11, the steps of a process for self-calibration 1100 during manufacturing or at the factory (also referred to as “pre-calibration”) are illustrated. Pre-calibration during manufacture is desirable to identify defective units of the wound therapy system and to configure the units with baseline performance at a therapy set point. Advantageously, slight variation in performance between various wound therapy devices can be compensated for during pre-calibration to ensure consistent performance of the devices when applied to patients.

The process 1000 can in block 1110 obtain the external pressure (referred herein to as “reference alpha” or “alpha reference pressure”). In connection with the device 100, the process 1000 can obtain one or more measurements from the second pressure sensor 1092 to determine reference alpha. Reference alpha can be used subsequently to determine changes in the external pressure and adjust operation.

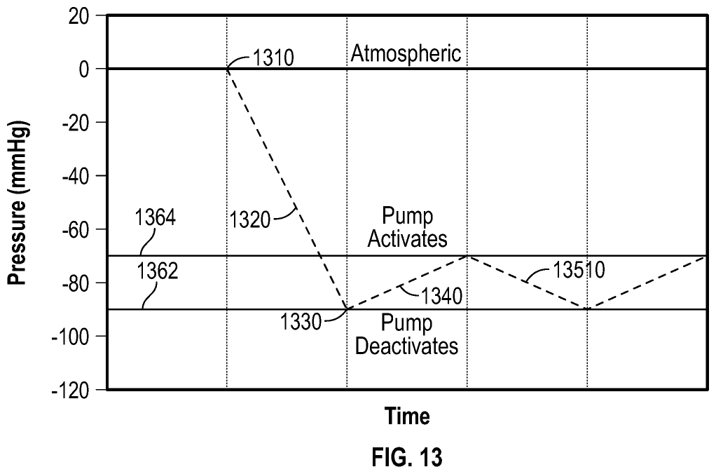

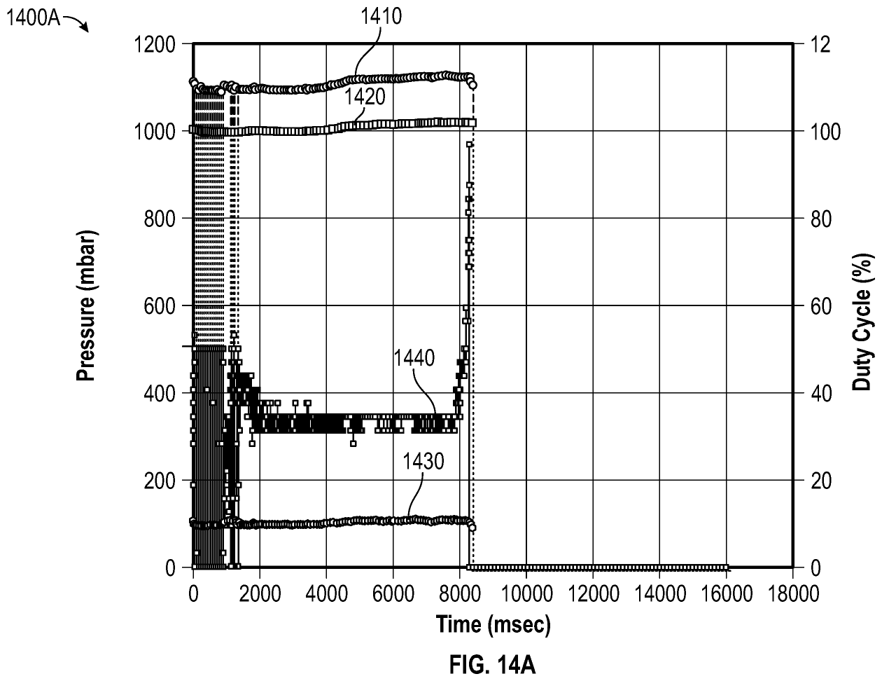

Having acquired the alpha reference pressure, the process 1100 can activate the negative pressure source to establish the negative pressure set point (also referred to as the therapy set point) in the fluid flow path of the wound therapy device. In manufacturing, the system may not be connected a real wound of a patient, but may be connected to a simulated wound (for instance, a wound model or another testing fixture). Accordingly, pressure in the fluid flow path (rather than at the wound) can be monitored by the process 1100. In some cases, as described herein, the process 1100 can be configured to alternate periods of activation and deactivation of the negative pressure source. For example, the process 1100 can operate the negative pressure source in the initial pump down (IPD) and maintenance pump down (MPD) modes (or states). With reference to FIG. 13, the process 1100 can be configured to make a transition from pressure in the fluid flow path being at the external pressure (which can, for example, occur in a standby state in which the negative pressure source is deactivated, as is illustrated by the segment 1310 in FIG. 13) to the IPD mode. In the IPD mode, the negative pressure source can be activated for delivery of therapy, as is illustrated by the segment 1320 in FIG. 13, in response to receiving a signal or automatically (such as, due to a timeout or due to initiation of the process 1100). Activation of the negative pressure source during IPD can cause the pressure in the fluid flow path to rapidly decrease (or become negative).

Responsive to achieving the negative pressure set point during IPD, the negative pressure source can be deactivated (illustrated as point 1330 in FIG. 13). In some cases, process 1100 can make a determination to deactivate the negative pressure source when the pressure in the fluid flow path reaches a first threshold pressure value (illustrated by 1362), which can equal to or exceed the negative pressure set point. The negative pressure set point can be approximately −80 mmHg, selected from the range of approximately −90 mmHg to −70 mmHg, or the like. The device can transition from the IPD mode to the MPD mode in response to the determination that the first threshold pressure value has been achieved.

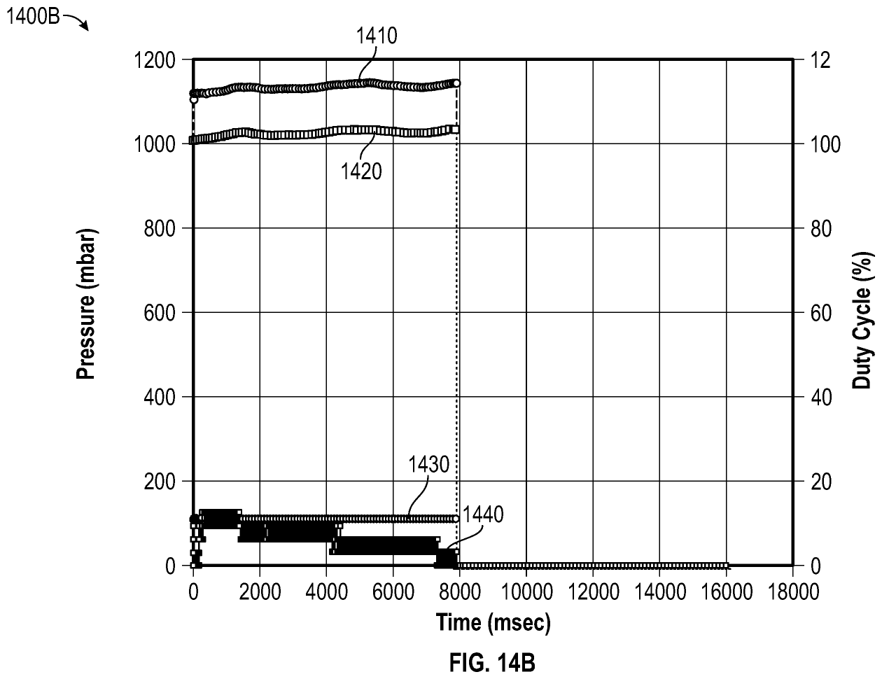

In block 1120 (which can be IPD), gas (such as, air) can be pumped out of the fluid flow path through the a non-return valve (such as, the non-return valve 1210). The process 1110 can control the intensity of operation of the negative pressure source (or the activity level of the negative pressure source), for instance, to ensure that the non-return valve opens and pressure is being lowered in the fluid flow path (see FIGS. 14A-14B and accompanying description). The process 1110 can control various activity parameters of the negative pressure source, such as, duty cycle (including number of pulses, timing of pulses, etc.), duration, power, speed, etc. For example, the control circuitry can initially activate the negative pressure source with repeated 100 millisecond pulses, then adjust one or more of the duration or duty cycle of the pulses to increase or decrease the activity level of the negative pressure source.

In block 1120, the process 1100 can continuously monitor the pressure in the fluid flow path in order to shut off the negative pressure source when a first threshold pressure value is reached (or transition to block 1250). In some cases, the monitoring can be performed by monitoring the pressure differential between the pressure sensors 1091 and 1092. In some cases, the process 1100 can use proportional-integral-derivative (PID) or proportional-integral (PI) control based on pressure feedback to control the negative pressure source in order to achieve the first threshold pressure value.

If the first threshold pressure value has been established in the fluid flow path, the process 1100 can transition to block 1130 where it records an activity level of the negative pressure source required to achieve the first threshold pressure value. The activity level can include one or more of the parameters disclosed herein (such as, duty cycle, duration, power, speed, duration, etc.). The recorded activity level can represent the worst case (or maximum) activity level required to establish the first threshold pressure value (which can include opening the non-return valve). This can be due to, in the IPD mode, having to pressure the fluid flow path from the external pressure to the first threshold pressure (such as, illustrated by the segment 1320 that can represent the steepest drop in pressure of all the segments illustrated in FIG. 13). The recorded activity level can be referred to as an “alpha pre-calibrated” activity level, which can be used as a baseline for operating the negative pressure source in the MPD mode during manufacturing and in the IPD and MPD modes when the wound therapy device has been installed on a patient.

The initial pump down (IPD) in block 1120 and recording of the activity level in block 1130 can be repeated during factory self-calibration to ensure reliable performance of the wound therapy device. In some cases, the wound therapy device can be configured to perform blocks 1120 and 1130 at least 5 times (or more or less) during factory self-calibration. In some cases, the recorded activity level 1130 can be an average of the activity levels measured during each iteration.

In certain instances, the wound therapy device may fail to achieve the first threshold pressure value in the IPD mode altogether. For instance, this can be due to a defective non-return valve. The process 1100 can transition to block 1150 where the device can be identified as defective. Such device can be flagged for additional testing.

Subsequent to establishing the first threshold pressure value in the IPD mode, the process 1100 can monitor the pressure in the fluid flow path (using any of the approaches described herein). Due to one or more leaks in the fluid flow path, negative pressure can be gradually lost in the fluid flow path. For example, there can be one or more leaks in a seal formed by a dressing, which can allow atmospheric air to gradually enter the fluid flow path. The loss of negative pressure can occur while the negative pressure source is deactivated following successfully completion of the IPD. This is illustrated in FIG. 13 by the segment 1340. To reestablish negative pressure in the fluid flow path, the process 1110 can perform one or more activations of the negative pressure source (referred to as a “maintenance pump down”). With reference to FIG. 13, in the MPD mode, the negative pressure source can be periodically activated to reestablish the first threshold pressure value. This is illustrated in FIG. 13 by the segment 1350 and other similar segments. As is illustrated in FIG. 13, negative pressure can be maintained in the range between first threshold pressure value 1362 (such as, −90 mmHg or less or more) and a second threshold pressure value 1364 (such as, −70 mmHg or less or more). A maintenance pump down can be triggered when the pressure in the fluid flow path reaches the second threshold pressure value 1364 (thereby indicating that the pressure in the fluid flow path has deviated significantly from the therapy set point due to one or more leaks). The maintenance pump down can be terminated once the first threshold pressure value has been reestablished.

With reference to FIG. 11, during a final stage of factory self-calibration, maintenance pump down (MPD) 1140 is used to test low-power operation of the wound therapy device. During the one or more maintenance pump downs in the MPD mode, the negative pressure source can be activated using a lower intensity than in the IPD mode (such as, a lower duty cycle or power level) to conserve the capacity of the power source. However, in some cases, process 1100 can utilize the alpha pre-calibrated activity level for one or more activations of the negative pressure source in the MPD mode since such activity level can represent the worst case scenario for ensuring that the first threshold pressure value would be established in the fluid flow path (including, for example, ensuring that the non-return valve opens). In the MPD mode, the process 1100 can continuously monitor the pressure in the fluid flow path and deactivate the negative pressure source once the first threshold pressure value has been reestablished.

In certain instances, the wound therapy device may fail to achieve the first threshold pressure value in the MPD mode altogether. The process 1100 can transition to block 1150 where the device can be identified as defective. Such device can be flagged for additional testing.

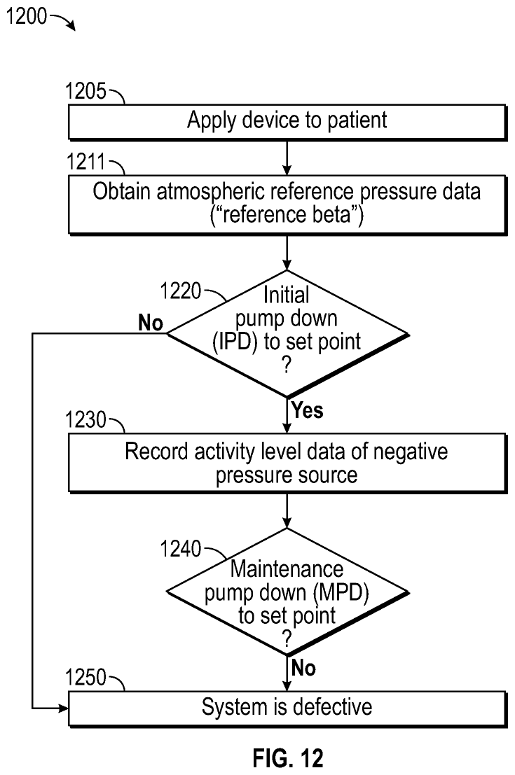

FIG. 12 illustrates a self-calibration process 1200 performed during application of negative pressure wound therapy to a wound of a patient The process 1200 can be similar to the process 1100 with the exception of the process 1200 being executed on the wound of the patient.

The process 1200 can be initiated responsive to the wound therapy device being applied to a patient in block 1205 In block 1205, negative pressure wound therapy can be initiated.

Similar to block 1110, the process 1200 can obtain in block 1211 the external pressure (referred herein to as “reference beta” or “beta reference pressure”). Reference beta can be used subsequently to determine changes in the external pressure and adjust operation. Similar to blocks 1120 and 1130, the process 1200 can determine and store “beta calibrated” activity level in blocks 1220 and 1230, which can be used as a baseline for operating the negative pressure source in the MPD mode. In some cases, a difference between blocks 1120 and 1220 can be that the process 1200 is executed when the device is treating the wound of the patient, and the process 1200 can determine beta calibrated activity level responsive to the first threshold pressure value having been established at the wound. In some cases, if in block 1120 the process 1200 is unable to establish the first threshold pressure value at the wound (or in the fluid flow path), alpha pre-calibrated activity level previously determined by the process 1100 can be utilized and recorded in block 1230.

In some instances, alpha pre-calibrated activity level can be scaled in block 1220. For example, if the beta reference pressure obtained in block 1211 is about 80% of the alpha reference pressure obtained in block 1110, alpha pre-calibrated activity level (such as, the duty cycle or duration) can be reduced by about 20%.

If the process 1200 is unable to establish the first threshold pressure value in block 1220, the process 1200 can transition to block 1250, which can be similar to the block 1150.

Similar to block 1140, in block 1240 the process 1200 can execute one or more maintenance pump downs using the beta calibrated activity level. If the first threshold pressure value cannot be reestablished, the process 1200 can transition to block 1250.

FIG. 14A illustrates a plot 1400A of operating a negative pressure wound therapy device in which the non-return valve fails to fully open during, for instance, maintenance pump down. Internal pressure sensor (such as, the pressure sensor 1091) measurements are illustrated at 1410. External pressure sensor (such as, the pressure sensor 1092) measurements are illustrated at 1420. Differential pressure measurements (such as, differential between 1420 and 1410) are illustrated at 1430. As described herein, differential pressure can be used to control the negative pressure source to establish or reestablish the first threshold pressure value. Activity level (such as, duty cycle) of the negative pressure source is illustrated at 1440. The x-axis illustrates time (in msec). The y-axis on the left side illustrate pressure (in mbar). The y-axis on the right side illustrate duty cycle (on percentage scale).

As is illustrated, external pressure sensor measurements 1420 are erratic. For example, while atmospheric pressure at sea level is 1013.25 mbar, external pressure sensor measurements 1420 are trending upward. This can be due to a non-return valve not being fully open during operation of the negative pressure source. Such erratic operation of the non-return valve can cause the external pressure sensor to detect incorrect pressure regardless of whether the pressure sensor is located upstream of downstream of the valve. If the external pressure sensor is located upstream of the valve (or between the negative pressure source and the valve), not fully open valve would prevent the sensor from correctly detecting the external pressure. If the external pressure sensor is located downstream of the valve, backpressure created by a not fully open valve would cause the sensor's pressure readings to be incorrect. The variation in the external pressure sensor measurement 1420 sensed can cause the differential pressure measurements 1430 to be unstable resulting in erratic changes in the activity level of the pump. The latter is illustrated by the duty cycle 1440 staying in a high range and even going up to around 100% (at about 8000 msec). This operation can undesirably drain the capacity of the power source.

FIG. 14B illustrates a plot 1400B of operating the negative pressure wound therapy device in which the non-return valve is fully open during, for instance, maintenance pump down. Unlike the plot 1400A, the external pressure sensor measurements 1420 remain relatively constant (for example, around 1013.25 mbar) causing the differential pressure measurements 1430 to remain stable. In the plot 1400B, the duty cycle 1440 remains in a much lower range than in the plot 1400A and steadily decreases to zero as the first threshold pressure value is reached (thus conserving the capacity of the power source). Self-calibration approaches described herein can help ensure that the device operates in accordance with the plot 1400B rather than the plot 1400A.

Self-calibration approaches described herein can improve performance of negative pressure wound therapy systems, particularly sealed systems which operate without a source of reference gas flow from the surrounding environment. Such systems can operate with two absolute pressure sensors (such as, pressure sensors 1091 and 1092) to measure a pressure differential between pressure at the wound and an external pressure reference or a differential pressure sensor that directly measures the pressure differential. Dynamic calibration and verification of the therapy performance can lessen (or eliminate) the risk of incorrect operation due to one or more changes in altitude or the variability of negative pressure sources, non-return valves, enclosures, tubing, connectors, filters, mechanical assemblies, or manufacturing. Such variability can be dynamically compensated for by self-calibration. Therapy performance (including stability of therapy, time of application of therapy, overall lifetime of therapy due to the conservation of capacity of the power source, or the like) and detection of one or more conditions (such as, leaks, blockages, overpressure, etc.) can be improved.

### Adjusting Operation Responsive to Atmospheric Pressure Changes

During operation of a wound therapy device, changes in the atmospheric pressure can be monitored and one or more operational parameters of the device can be adjusted. In some instances, particularly when the device is being used aboard an aircraft, the device must react to rapid changes in the atmospheric pressure (which may be caused by changes in the altitude) to lessen or avoid the risk of providing excessive negative pressure to the patient. This section describes approaches for adjusting one or more operational parameters responsive to changes in atmospheric pressure (which may be caused by altitude changes) for improved patient comfort and safety.

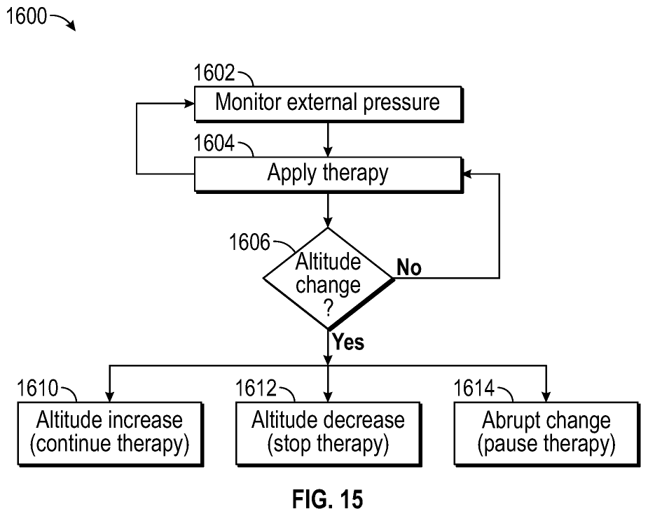

FIG. 15 illustrates a process 1600 for responding to changes in the external pressure (such as, atmospheric pressure). The process 1600 can be implemented by any one or more of controllers or processors of negative pressure wound therapy devices described herein. In block 1602, the process 1600 can monitor the external pressure (for instance, similarly to blocks 1110 and 1211). This monitoring can be performed when a negative pressure source in deactivated (to obtain accurate measurement of the external pressure). The monitoring in block 1602 can be continuous (such as, prior to the IPD, in between maintenance pump down cycles, or the like), as indicated by the arrows forming a loop between block 1602 and 1604). In block 1604, the process 1600 can activate the negative pressure source to apply negative pressure wound therapy to a wound of a patient.

In block 1606, the process can determine whether there has been a change in altitude relative to prior operation of the device. Changes in the altitude can be determined through changes in the atmospheric pressure (since atmospheric pressure decreases with increasing elevation), which can be monitored by the process 1600 using any of the approaches described herein. For example, external pressure obtained in block 1602 can be compared to a reference pressure previously recorded, such as the beta reference pressure. External pressure obtained in block 1602 can be recorded as the reference pressure for subsequent execution of the process 1600. In block 1606, the process can determine whether the attitude change satisfies one or more thresholds. The one or more thresholds can be selected as described below for block 1614. For instance, the one or more thresholds can be selected to distinguish the situation of the patient being in an aircraft during climb or descent as opposed to the patient being in a vehicle that travels up or down a mountain. If not, the process 1600 can transition back to block 1604. If yes, the process 1600 can determine whether there has been one or more of an increase (block 1610), decrease (block 1612), or abrupt change in the altitude (block 1614).

If the process 1600 has determined that the altitude is increasing (or the atmospheric pressure is decreasing, such as, during aircraft takeoff), the process can transition to block 1610 where it can continue application of negative pressure wound therapy. Activity of the negative pressure source (such as, the duty cycle) can be reduced to conserve the capacity of the power source (see FIG. 14A and accompanying description), while maintaining at least some level of negative pressure at the wound. In some cases, the reduction of the activity would naturally take place since atmospheric pressure is decreasing as the altitude is increasing (thereby causing the pressure differential between pressure at the wound and external pressure to decrease). Leakage (or blockage or another type of) alarm or alert can be suppressed since reaching the negative pressure set point is delayed due to the change in altitude, not due to a leak (or blockage). External pressure can be monitored to determine when the altitude has stabilized and activity of the negative pressure source can be returned to the normal activity level associated with maintaining the negative pressure set point. Determination of whether the altitude has stabilized can be performed over a period of time, such as 40 minutes or less or more.

If the process 1600 has determined that the altitude is decreasing (or the negative pressure is increasing, such as, during aircraft landing), the process can transition to block 1612 where it can stop application of negative pressure wound therapy (such as, by deactivating the negative pressure source) to facilitate patient safety. Decrease in altitude would lead to increase in the atmospheric pressure, causing the pressure differential to increase and potentially leading to excessive negative pressure being applied to the wound. Any excess negative pressure can be allowed to dissipate through normal leak(s) of the wound dressing. Additionally or alternatively, a valve can be opened to release any excess negative pressure. In block 1612, blockage (or excessive vacuum, leakage, or another type of) alarm or alert can be suppressed since therapy has been stopped due to the change in altitude, not due to a blockage (or excessive vacuum or leakage). External pressure can be monitored to determine when the altitude has stabilized and therapy can be restarted.

In the process 1600 has determined that the altitude has changed abruptly (either decreased or increased), the process can transition to block 1614 where it can pause application of the negative pressure wound therapy. Determination of whether altitude has changed abruptly can be performed based on comparing the altitude (or pressure) change to one or more thresholds indicative of an abrupt change. For example, one such threshold can correspond to a rate of change in cabin pressure altitude during climb of a commercial aircraft (in which the cabin is pressurized), which can be limited to no more than about 5 m/s (meters per second) sea-level equivalent to ensure passenger comfort. As another example, another such threshold can correspond to a rate of change in cabin pressure altitude during descent of the commercial aircraft, which can be limited to no more than about 2.3 m/s sea-level equivalent. Other suitable thresholds can be used for climb and descent of military aircraft or rate of altitude (or pressure) change in a helicopter (in which the cabin may not be pressured). The one or more thresholds for determining abrupt change in the attitude can be selected to distinguish from a situation in which the patient is in a vehicle that is driving up or down a mountain during which the atmospheric pressure is not changing abruptly.

Block 1614 can be implemented to protect the patient from discomfort (such as, pain) or injury (such as, bleeding) caused by overpressure (in case of an abrupt decrease in the altitude) or conserve the capacity of the power source (in case of an abrupt increase in the altitude). The process 1600 can maintain therapy delivery in a paused state until the altitude has stabilized.

In some cases, block 1614 may be optional as blocks 1610 and 1612 may be sufficient. In some implementations, blocks 1610 and 1612 may be optional as block 1614 may be sufficient.

In some cases, one or more additional sensors that directly measure altitude changes (such as, accelerometer, magnetometer, gyrometer, altimeter, etc.) can be additionally or alternatively used to monitor altitude changes.

Advantageously, the approaches described in this section can help negative pressure wound therapy devices to compensate for changes in the operating environment and allow therapy to be applied with fewer (or no) interruptions while promoting patient safety.

### Other Variations

While certain embodiments described herein relate to integrated negative pressure wound therapy systems in which the negative pressure source is supported by the dressing, systems and methods described herein are applicable to any negative pressure wound therapy system or medical system, particularly to systems being positioned on (or worn by) the patient. For example, systems and methods for controlling operation described herein can be used in fluid-proof (such as, water-proof) negative pressure wound therapy systems or medical systems. Such systems can be configured with the negative pressure source and/or electronics being external to the wound dressing, such as with the negative pressure source and/or electronics being positioned in a fluid proof enclosure. Additionally, such systems can be configured to be used within ultrasound delivery devices, negative pressure devices powered by an external power supply, negative pressure devices with a separate pump, and medical devices generally. In some cases, the systems and methods described herein are applicable to negative pressure wound therapy systems that utilize an absolute pressure sensor to measure pressure at the wound and another absolute pressure sensor to measure a reference atmospheric pressure. Such negative pressure wound therapy systems can be sealed so that there is no deliberate mechanism for admitting a controlled flow of gas from the external environment into the fluid flow path (which is sometimes referred to as a controlled leak).

Any of the embodiments disclosed herein can be used with one or more features disclosed in U.S. Pat. No. 7,779,625, titled “DEVICE AND METHOD FOR WOUND THERAPY,” issued Aug. 24, 2010; U.S. Pat. No. 7,964,766, titled “WOUND CLEANSING APPARATUS IN SITU,” issued on Jun. 21, 2011; U.S. Pat. No. 8,235,955, titled “WOUND TREATMENT APPARATUS AND METHOD,” issued on Aug. 7, 2012; U.S. Pat. No. 7,753,894, titled “WOUND CLEANSING APPARATUS WITH STRESS,” issued Jul. 13, 2010; U.S. Pat. No. 8,764,732, titled “WOUND DRESSING,” issued Jul. 1, 2013; U.S. Pat. No. 8,808,274, titled “WOUND DRESSING,” issued Aug. 19, 2013; U.S. Pat. No. 9,061,095, titled “WOUND DRESSING AND METHOD OF USE,” issued Jun. 23, 2015; U.S. Pat. No. 10,076,449, issued Sep. 18, 2018, titled “WOUND DRESSING AND METHOD OF TREATMENT”; U.S. patent application Ser. No. 13/418,908, filed Jan. 30, 2015, published as U.S. Publication No. 2015/0190286, published Jul. 9, 2015, titled “WOUND DRESSING AND METHOD OF TREATMENT”; U.S. Pat. No. 10,231,878, titled “TISSUE HEALING,” issued Mar. 19, 2019; PCT International Application PCT/GB2012/000587, titled “WOUND DRESSING AND METHOD OF TREATMENT” and filed on Jul. 12, 2012; International Application No. PCT/IB2013/001369, filed May 22, 2013, titled “APPARATUSES AND METHODS FOR NEGATIVE PRESSURE WOUND THERAPY”; PCT International Application No. PCT/IB2013/002102, filed Jul. 31, 2013, titled “WOUND DRESSING AND METHOD OF TREATMENT”; PCT International Application No. PCT/IB2013/002060, filed Jul. 31, 2013, titled “WOUND DRESSING AND METHOD OF TREATMENT”; PCT International Application No. PCT/IB2013/00084, filed Mar. 12, 2013, titled “REDUCED PRESSURE APPARATUS AND METHODS”; International Application No. PCT/EP2016/059329, filed Apr. 26, 2016, titled “REDUCED PRESSURE APPARATUSES”; PCT International Application No. PCT/EP2017/059883, filed Apr. 26, 2017, titled “WOUND DRESSINGS AND METHODS OF USE WITH INTEGRATED NEGATIVE PRESSURE SOURCE HAVING A FLUID INGRESS INHIBITION COMPONENT”; PCT International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING”; PCT International Application No. PCT/EP2018/074694, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/074701, filed Sep. 13, 2018, titled “NEGATIVE PRESSURE WOUND TREATMENT APPARATUSES AND METHODS WITH INTEGRATED ELECTRONICS”; PCT International Application No. PCT/EP2018/079345, filed Oct. 25, 2018, titled “NEGATIVE PRESSURE WOUND TREATMENT APPARATUSES AND METHODS WITH INTEGRATED ELECTRONICS”; PCT International Application No. PCT/EP2018/079745, filed Oct. 30, 2018, titled “SAFE OPERATION OF INTEGRATED NEGATIVE PRESSURE WOUND TREATMENT APPARATUSES”; each of which is incorporated by reference herein in its entirety.

Although certain embodiments described herein relate to wound dressings, systems and methods disclosed herein are not limited to wound dressings or medical applications. Systems and methods disclosed herein are generally applicable to electronic devices in general, such as electronic devices that can be worn by or applied to a user.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure.

The various components illustrated in the figures or described herein may be implemented as software or firmware on a processor, controller, ASIC, FPGA, or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICS, FPGAs, and the like, can include logic circuitry. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A negative pressure wound therapy system comprising:
a wound dressing configured to be placed over a wound and absorb fluid aspirated from the wound;
a negative pressure source configured to aspirate fluid from the wound via a fluid flow path connecting the negative pressure source to the wound dressing;
a non-return valve positioned in the fluid flow path and fluidically connected to the negative pressure source; and
an electronic control circuitry configured to:
based on a pressure difference between a first pressure in the fluid flow path and a second pressure of an environment surrounding the wound, activate the negative pressure source to establish a target negative pressure level in the fluid flow path;
determine an activity level of the negative pressure source required to open and maintain the non-return valve in an opened state and establish the target negative pressure level in the fluid flow path; and
use the activity level during one or more subsequent activations of the negative pressure source to establish the target negative pressure level in the fluid flow path.

2. The negative pressure wound therapy system of claim 1, wherein the electronic control circuitry is configured to:
determine the activity level of the negative pressure source responsive to initially establishing the target negative pressure level in the fluid flow path; and
use the activity level for maintaining the target negative pressure level in the fluid flow path following initially establishing the target negative pressure level in the fluid flow path.

3. The negative pressure wound therapy system of claim 2, wherein the electronic control circuitry is configured to deactivate the negative pressure source responsive to initially establishing the target negative pressure level in the fluid flow path and subsequently activate the negative pressure source responsive to negative pressure in the fluid flow path becoming more positive than the target negative pressure level.

4. The negative pressure wound therapy system of claim 1, wherein the electronic control circuitry is further configured to:
monitor a change in the second pressure of the environment surrounding the wound; and
responsive to determining that the second pressure is increasing, deactivate the negative pressure source to prevent reducing pressure at the wound to a negative pressure level associated with unsafe negative pressure.

5. The negative pressure wound therapy system of claim 4, wherein the electronic control circuitry is further configured to:
responsive to determining that the second pressure is decreasing, reduce the activity level of the negative pressure source until the second pressure of the environment surrounding the wound has stabilized.

6. The negative pressure wound therapy system of claim 4, wherein the electronic control circuitry is further configured to:
responsive to determining that the change in the second pressure satisfies a threshold indicative or an abrupt change in pressure, deactivate the negative pressure source until the second pressure has stabilized.

7. The negative pressure wound therapy system of claim 4, wherein the electronic control circuitry is configured to record a reference value of the second pressure responsive to activating the negative pressure source to establish the target negative pressure level in the fluid flow path and use the reference value of the second pressure to monitor the change in the second pressure.

8. The negative pressure wound therapy system of claim 1, further comprising:
a first absolute pressure sensor configured to monitor the first pressure in the fluid flow path; and
a second absolute pressure sensor configured to monitor the second pressure of the environment surrounding the wound,
wherein the electronic control circuitry is configured to determine the pressure difference based on the first pressure monitored by the first absolute pressure sensor and the second pressure monitored by the second absolute pressure sensor.

9. The negative pressure wound therapy system of claim 1, wherein at least one of the negative pressure source or the electronic control circuitry is disposed on or within the wound dressing.

10. The negative pressure wound therapy system of claim 1, further comprising a differential pressure sensor configured to monitor the pressure difference between the first pressure in the fluid flow path and the second pressure of the environment surrounding the wound, wherein the differential pressure sensor does not reference atmospheric pressure.

11. The negative pressure wound therapy system of claim 1, wherein the electronic control circuitry is configured to determine the activity level responsive to calibration of the system and use the activity level for aspirating fluid from the wound of a patient.

12. The negative pressure wound therapy system of claim 11, wherein calibration is performed during manufacturing.

13. (canceled)

14. The negative pressure wound therapy system of claim 1, further comprising a power source configured to provide power to the negative pressure source and the electronic control circuitry, wherein the electronic control circuitry is configured to operate the negative pressure source by alternately activating and deactivating the negative pressure source to preserve capacity of the power source.

15. A method of operating a negative pressure wound therapy system, the method comprising, by an electronic control circuitry of the system:
based on a pressure difference between a first pressure in a fluid flow path connecting a negative pressure source of the system to a wound covered by a wound dressing and a second pressure of an environment surrounding the wound, activating the negative pressure source to establish a target negative pressure level in the fluid flow path;
determining an activity level of the negative pressure source required to open and maintain in an open state a non-return valve of the system and establish the target negative pressure level in the fluid flow path; and
using the activity level, establishing the target negative pressure level in the fluid flow path during one or more subsequent activations of the negative pressure source.

16. The method of any of claim 15, further comprising:
determining the activity level of the negative pressure source responsive to initially establishing the target negative pressure level in the fluid flow path; and
using the activity level for maintaining the target negative pressure level in the fluid flow path following initially establishing the target negative pressure level in the fluid flow path.

17. The method of claim 15, further comprising:
monitoring a change in the second pressure of the environment surrounding the wound; and
responsive to determining that the second pressure is increasing, deactivating the negative pressure source to prevent reducing pressure at the wound to a negative pressure level associated with unsafe negative pressure.

18. The method of claim 17, further comprising:
responsive to determining that the second pressure is decreasing, reducing the activity level of the negative pressure source until the second pressure of the environment surrounding the wound has stabilized.

19. The method of claim 17, further comprising:
responsive to determining that the change in the second pressure satisfies a threshold indicative or an abrupt change in pressure, deactivating the negative pressure source until the second pressure has stabilized.

20. The method of claim 17, further comprising recording a reference value of the second pressure responsive to activating the negative pressure source to establish the target negative pressure level in the fluid flow path and using the reference value of the second pressure to monitor the change in the second pressure.

21. The method of claim 17, wherein determining the activity level is performed responsive to calibration of the system during manufacturing, and wherein the method further comprises using the activity level for aspirating fluid from the wound of a patient.

22. (canceled)
